(19) United States
(12) Patent Application Publication
(10) Pub. No.: US 2014/0296682 A1
(43) Pub. Date: Oct. 2, 2014

(54) BIOLOGICAL-INFORMATION ACQUISITION APPARATUS AND BIOLOGICAL-INFORMATION COMMUNICATION SYSTEM

(71) Applicant: Sony Corporation, Tokyo (JP)

(72) Inventors: Seiji Wada, Kanagawa (JP); Takashi Tomita, Kanagawa (JP); Hirotaka Muramatsu, Tokyo (JP)

(73) Assignee: Sony Corporation, Tokyo (JP)

(21) Appl. No.: 14/222,155

(22) Filed: Mar. 21, 2014

(30) Foreign Application Priority Data
Mar. 29, 2013 (JP) 2013-071907

(51) Int. Cl. A61B 5/0408

(52) U.S. Cl. CPC A61B 5/04085
USPC 600/384; 600/386; 600/391

(57) ABSTRACT

There is provided a biological-information acquisition apparatus including a plurality of flexible attachment devices each provided with an electrode that is attached to a body and that is configured to acquire biological information, and a connector configured to connect the plurality of attachment devices.

### CROSS REFERENCE TO RELATED APPLICATIONS

This application claims the benefit of Japanese Priority Patent Application JP 2013-071907 filed Mar. 29, 2013, the entire contents of which are incorporated herein by reference.

### BACKGROUND

The present disclosure relates to biological-information acquisition apparatuses and biological-information communication systems.

In the related art, for example, JP H3-128040A discusses biological electrodes in which electrodes are disposed on a base material to be attached to biological measurement sites.

JP 2012-235565A discusses a transmission system in which a biological information sensor (i.e., a responding apparatus) is driven by being supplied with electric power from an information processing apparatus (i.e., an inquiring apparatus) such that the sensor side is passive.

### SUMMARY

However, although the base material having the electrodes attached thereto is a jacket in the technology discussed in JP H3-128040A, a method of how the base material is attached to or detached from a body is not taken into consideration. Therefore, it is difficult for a subject (i.e., a patient) to readily attach the base material provided with the electrodes to his/her body by himself/herself. In addition, it is difficult for the subject to attach the electrodes to proper positions by himself/herself when, for example, acquiring electrocardiographic waveforms.

In the technology discussed in JP 2012-235565A, the power supply time varies in accordance with the communication environment between the inquiring apparatus and the responding apparatus, which is a problem in that the time it takes for the inquiring apparatus to sample biological information varies. Thus, it is sometimes difficult for the responding apparatus to acquire the biological information at an appropriate timing. Accordingly, in the transmission system in which the biological information sensor is driven by being supplied with electric power from the information processing apparatus such that the sensor side is passive, a change in the time taken to supply electric power to the biological information sensor causes sampling intervals to fluctuate, thus making it difficult to handle biological information in which accurate sampling intervals are demanded.

Thus, it is demanded that the subject can readily attach the electrodes to proper positions by himself/herself. In addition, in a system that supplies electric power to an apparatus that acquires biological information, it is demanded that the biological information be acquired at appropriate sampling intervals.

According to an embodiment of the present disclosure, there is provided a biological-information acquisition apparatus including a plurality of flexible attachment devices each provided with an electrode that is attached to a body and that is configured to acquire biological information, and a connector configured to connect the plurality of attachment devices.

Further, one of the attachment devices may be attached to a chest area and acquires an electrocardiographic chest-lead waveform as the biological information.

Further, one of the attachment devices may be attached to a right arm or a left arm and acquires an electrocardiographic limb-lead waveform as the biological information.

Further, one of the attachment devices is attached to a hip and acquires an electrocardiographic limb-lead waveform as the biological information.

Further, the biological-information acquisition apparatus may further include a main device configured to acquire the biological information from each of the attachment devices and transmit the biological information to a communication apparatus via intra-body communication.

Further, the main device may be connected to one of the attachment devices via the connector.

Further, the communication apparatus may transmit the biological information to an electronic apparatus configured to determine whether each electrode is in an attached state based on the biological information.

Further, the electronic apparatus may include a display unit configured to display a guide for attaching the attachment devices to the body.

Further, each electrode may be formed by laminating an adhesive layer attachable to the body, a first conductive layer, an electrolyte layer, and a second conductive layer in this order, and a predetermined potential difference is applied between the first conductive layer and the second conductive layer when the electrode is to be detached from the body.

Further, the electrolyte layer and the adhesive layer may be each composed of a polyethylene-ethylene-oxide-hexamethylene copolymer or SBR polyethylene-oxide copolymer impregnated with an ionic material.

Further, the first conductive layer and the second conductive layer may be each formed of a carbon fiber layer.

Further, the first conductive layer has a foamable solid material mixed therein.

Further, according to an embodiment of the present disclosure, there is provided a communication system including a biological-information acquisition apparatus including an electrode that is attached to a body and that is configured to acquire biological information, a transmitting unit configured to transmit the biological information acquired by the electrode, and a power receiving unit configured to receive supplied electric power, and an information processing apparatus including a power supply unit configured to perform power supply to the biological-information acquisition apparatus via intra-body communication, a receiving unit configured to receive the biological information from the transmitting unit via intra-body communication, a sampling-interval determination unit configured to determine a sampling interval extending from when the power supply commences to when the biological information is received, and an interpolation unit configured to interpolate biological information in the sampling interval and acquire the biological information in a case where the sampling interval is deviated from a predetermined value.

According to one or more of embodiments of the present disclosure, the subject can readily attach the electrodes to proper positions by himself/herself. In addition, in a system that supplies electric power to an apparatus that acquires biological information, biological information can be acquired at appropriate sampling intervals.

### BRIEF DESCRIPTION OF THE DRAWINGS

FIG. 1 schematically illustrates electrode positions and output waveforms;

FIG. 2 schematically illustrates a state where a biological-information acquisition apparatus is attached to a human body;

FIG. 3 is a block diagram illustrating the configuration of a system including the biological-information acquisition apparatus;

FIG. 4 schematically illustrates the configuration of an electrode attachment device and electrode attachment positions according to an embodiment;

FIG. 5 schematically illustrates a method of how the electrode attachment device is attached;

FIG. 6 is a flowchart illustrating a process for checking that electrodes are properly attached;

FIG. 7 schematically illustrates a problem detection method based on lead waveforms;

FIG. 8 illustrates a specific example of problem detection based on the lead waveforms;

FIG. 9 illustrates a specific example of problem detection based on the lead waveforms;

FIG. 10 illustrates a specific example of problem detection based on the lead waveforms;

FIG. 11 illustrates a specific example of problem detection based on the lead waveforms;

FIG. 12 illustrates a specific example of problem detection based on the lead waveforms;

FIG. 13 is a flowchart illustrating a problem detection process based on FIGS. 7 to 12;

FIG. 14 schematically illustrates the electrode attachment device applied to an observational 18-lead electrocardiograph;

FIG. 15 schematically illustrates a schematic configuration of a system according to a second embodiment of the present disclosure;

FIG. 16 schematically illustrates the system including an inquiring apparatus and responding apparatuses;

FIG. 17 is a schematic functional block diagram of a control unit of the inquiring apparatus;

FIG. 18 is a characteristic diagram expressing the relationship between reception voltage and time in a power receiving unit of each responding apparatus;

FIG. 19 is a sequence diagram illustrating the operation of the inquiring apparatus and each responding apparatus;

FIG. 20 illustrates an interpolation process performed in an interpolation unit and is a characteristic diagram showing time-series data of biological information in a certain period;

FIG. 21 is a schematic cross-sectional view illustrating the configuration of an electrode according to a third embodiment; and

FIG. 22 is a schematic cross-sectional view illustrating another example of an electrode according to the third embodiment.

### DETAILED DESCRIPTION OF THE EMBODIMENTS

Hereinafter, preferred embodiments of the present disclosure will be described in detail with reference to the appended drawings. Note that, in this specification and the appended drawings, structural elements that have substantially the same function and structure are denoted with the same reference numerals, and repeated explanation of these structural elements is omitted.

The description below will proceed in the following order.

### 1. First Embodiment

1.1. General Outline of Biological-Information Acquisition According to First Embodiment

1.2. Configuration Example of System Including Biological-Information Acquisition Apparatus

1.3. Configuration Example of Electrode Attachment Device

1.4. Method for Attaching Electrode Attachment Device

1.5. Process for Checking that Electrodes are Properly Attached

1.6. Example of Application to Observational 18-Lead Electrocardiograph

### 2. Second Embodiment

2.1. Configuration Example of System According to Second Embodiment

2.2. Operation Sequence of Inquiring Apparatus and Responding Apparatus

2.3. Interpolation Process by Interpolation Unit

### 3. Third Embodiment

3.1. Configuration Example of Electrode According to Third Embodiment

3.2. Method for Manufacturing Electrolyte Layer

### 1. First Embodiment

### 1.1. General Outline of Biological-Information Acquisition According to First Embodiment

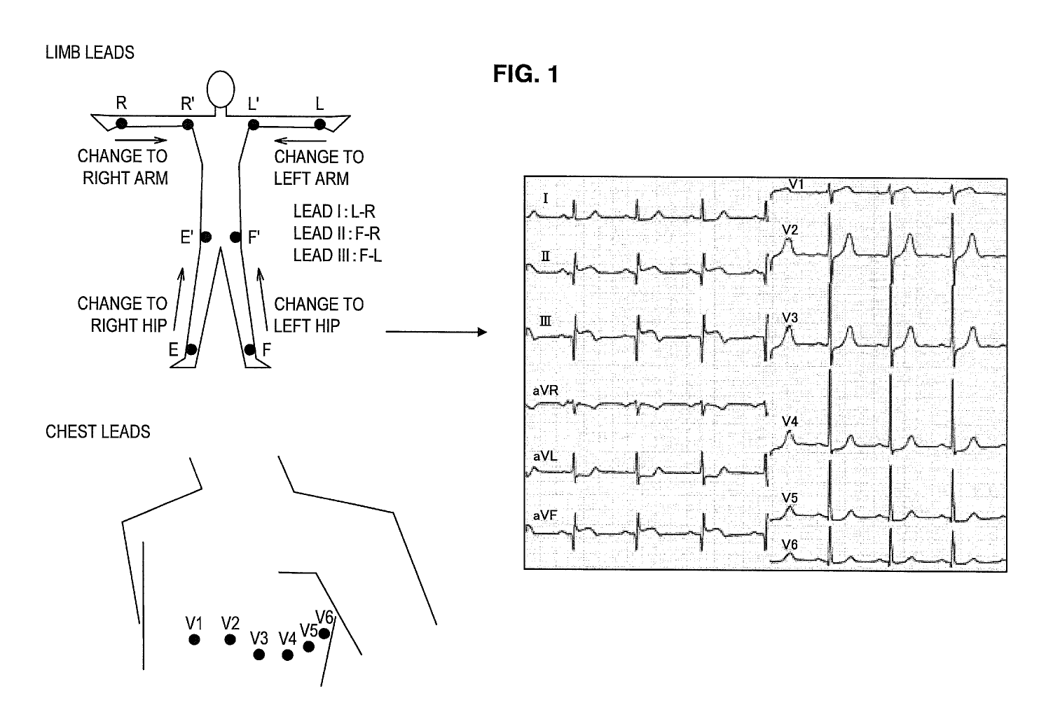

In this embodiment, a system that acquires an electrocardiogram by using a biological-information acquisition apparatus 100 will be described. A 12-lead electrocardiogram generally used for diagnosing and treating a heart disease outputs twelve kinds of waveforms that are obtained by attaching electrodes to ten positions of a human body. FIG. 1 schematically illustrates electrode positions and output waveforms. Reference characters R, L, F, E, V1, V2, V3, V4, V5, and V6 at the left side of FIG. 1 denote electrodes attached to the body, and a characteristic diagram shown at the right side of FIG. 1 illustrates the twelve kinds of waveforms. As shown in FIG. 1, the electrodes are attached to four limb-lead positions (i.e., R, L, F, and E) and six chest-lead positions (i.e., V1, V2, V3, V4, V5, and V6). Among the four limb leads, the electrode E serves as a ground potential. The twelve kinds of waveforms include waveforms detected by the electrodes as well as waveforms derived from the waveforms detected by the electrodes. As will be described later, with regard to the four limb-lead positions (i.e., R, L, F, and E) in this embodiment, the electrode attachment positions are changed to R′, L′, F′, and E′.

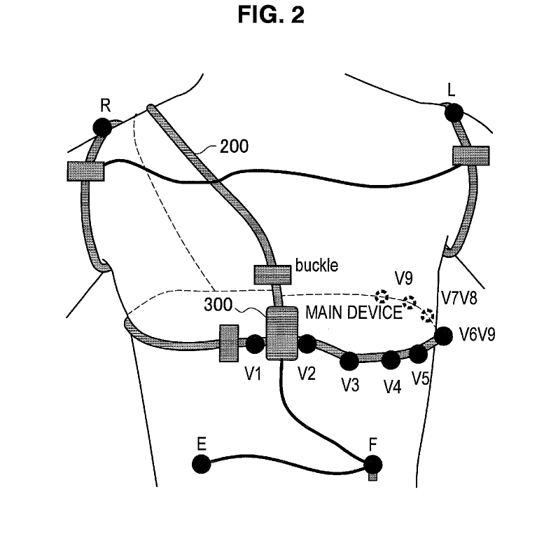

FIG. 2 illustrates a state where the biological-information acquisition apparatus 100 is attached to the human body, and shows the state of the upper half of the human body. The biological-information acquisition apparatus 100 includes an electrode attachment device 200 and a main device 300. As shown in FIG. 2, in this embodiment, the electrode positions of the biological-information acquisition apparatus 100 are set in correspondence with electrode attachment positions normally used with 12-lead electrocardiograms, and the biological-information acquisition apparatus 100 is attached to the torso or to an area near the torso. Therefore, the electrodes R and L shown in FIG. 2 are attached to the shoulders, and the electrodes F and E are attached to the abdomen. The electrodes V1, V2, V3, V4, V5, and V6 are attached to the chest area, as usual.

### 1.2. Configuration Example of System Including Biological-Information Acquisition Apparatus

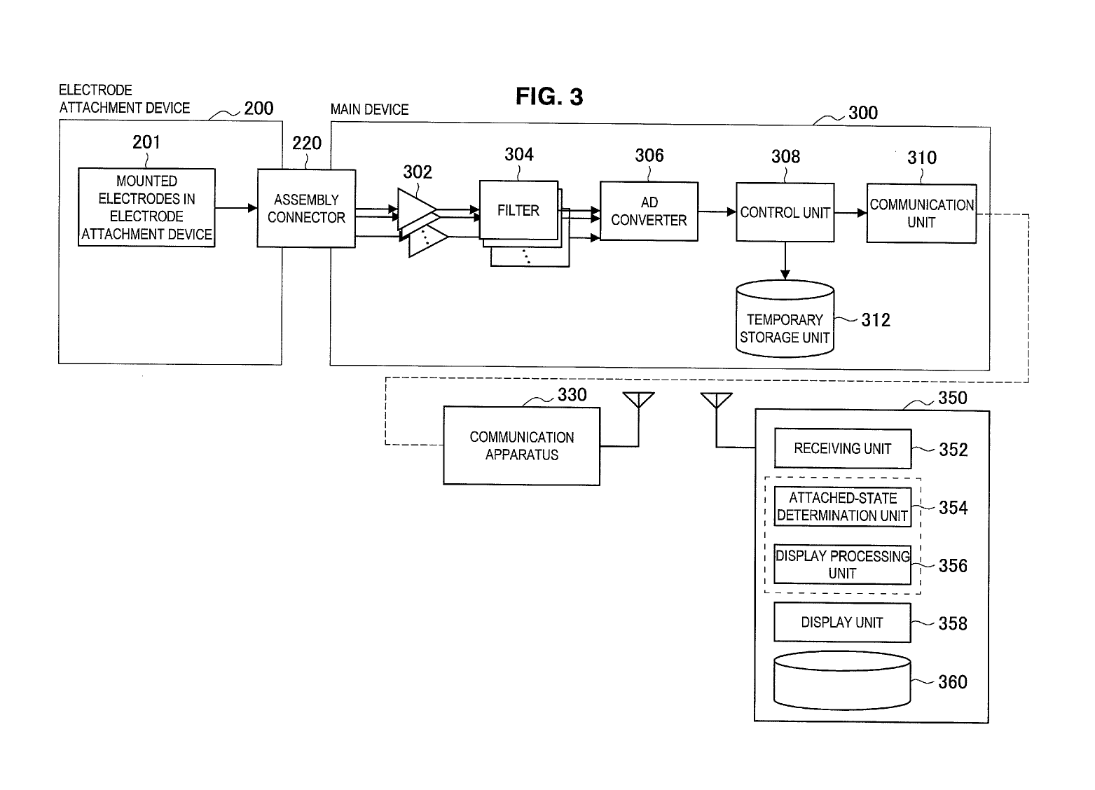

FIG. 3 is a block diagram illustrating the configuration of a system including the biological-information acquisition apparatus 100. As shown in FIG. 3, this system includes the biological-information acquisition apparatus 100, a communication apparatus 330, and an electronic apparatus 350. The electrode attachment device 200 and the main device 300 are separated from each other, and are connected to each other via an assembly connector 220. As shown in FIG. 3, the electrode attachment device 200 includes a plurality of electrodes 201. The multiple electrodes 201 correspond to the electrodes R, L, F, E, V1, V2, V3, V4, V5, and V6. The main device 300 includes an amplifier 302, a filter 304, an analog-to-digital (AD) converter 306, a control unit 308, a communication unit 310, and a storage unit 312. The amplifier 302 amplifies a signal waveform detected by each electrode 201 of the electrode attachment device 200. The filter 304 performs filtering for removing, for example, noise from the amplified signal waveform. The AD converter 306 converts an analog signal output from the filter 304 into a digital signal. The control unit 308 serves as a component that controls the main device 300 and performs processing, such as generating a lead waveform by performing an arithmetic process on the input digital signal. The storage unit 312 stores the signal transmitted from the control unit 308. The communication unit 310 transmits the signal transmitted from the control unit 308 to the communication apparatus 330.

The communication apparatus 330 is communicable with the biological-information acquisition apparatus 100 via, for example, intra-body communication. The communication apparatus 330 receives the signal waveform transmitted from the biological-information acquisition apparatus 100 and transmits the signal waveform to the electronic apparatus 350, which is an external apparatus such as a personal computer, a tablet terminal, or a portable telephone.

The electronic apparatus 350 includes a receiving unit 352 that receives the signal transmitted from the communication apparatus 330, an attached-state determination unit 354 that determines whether each electrode 201 is in an attached state on the basis of the received signal, a display processing unit 356 that performs processing for displaying, for example, the attached state of each electrode 201, the signal waveform received from the biological-information acquisition apparatus 100, and an attachment guide, a display unit (liquid crystal display (LCD)) 358 that performs display on the basis of the processing performed by the display processing unit 356, and a database 360 that stores, for example, the attachment guide.

### 1.3. Configuration Example of Electrode Attachment Device

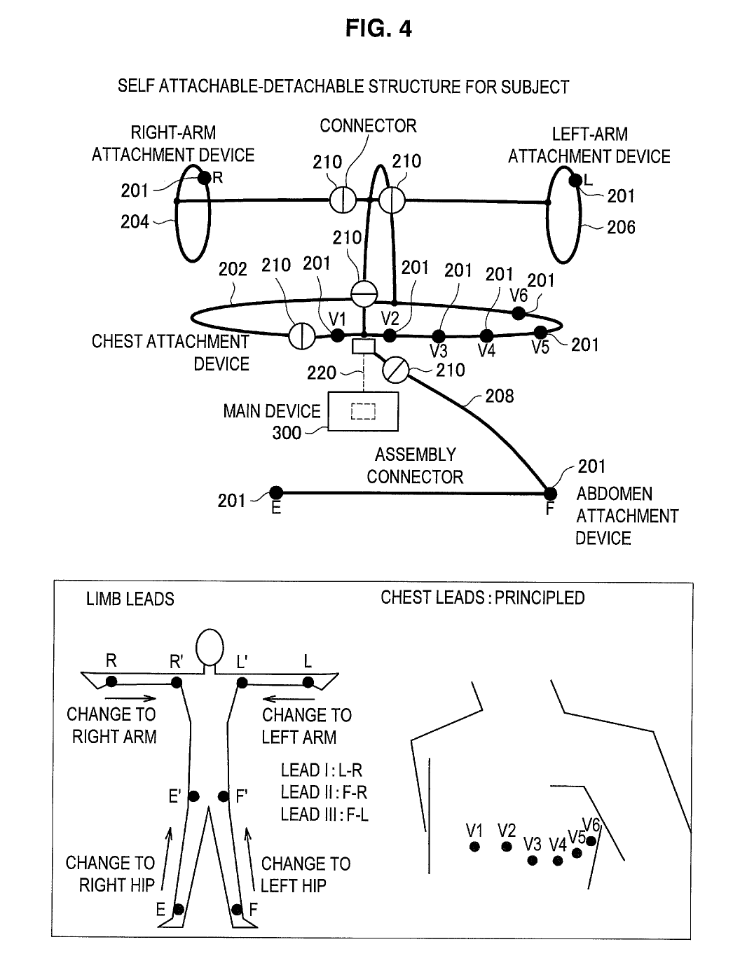

FIG. 4 schematically illustrates the configuration of the electrode attachment device 200 and the electrode attachment positions according to this embodiment. The electrode attachment device 200 is divided into a chest attachment device 202, a right-arm attachment device 204, a left-arm attachment device 206, an abdomen attachment device 208, and the main device 300. Cables extending from the electrodes 201 included in the individual attachment devices are relayed by connectors 210 interposed between the attachment devices and are gathered at the assembly connector 220 for connecting the main device 300 thereto. The assembly connector 220 is provided at the chest position corresponding to the chest attachment device 202.

As shown in FIG. 4, for the chest leads, the electrodes are attached to predetermined principled positions. For the limb leads, the attachment position of the electrode L is changed from the usual left wrist position to a left arm (left shoulder) position. The attachment position of the electrode R is changed from the usual right wrist position to a right arm (right shoulder) position. The attachment position of the electrode F is changed from the usual left ankle position to a left hip position, and the attachment position of the electrode E is changed from the usual right ankle position to a right hip position. In other words, the four limb-lead positions (i.e., R, L, F, and E) are changed to the positions of electrodes R′, L′, F′, and E′. With this positional change, the electrode attachment device 200 can be attached only to the upper half of the body.

### 1.4. Method for Attaching Electrode Attachment Device

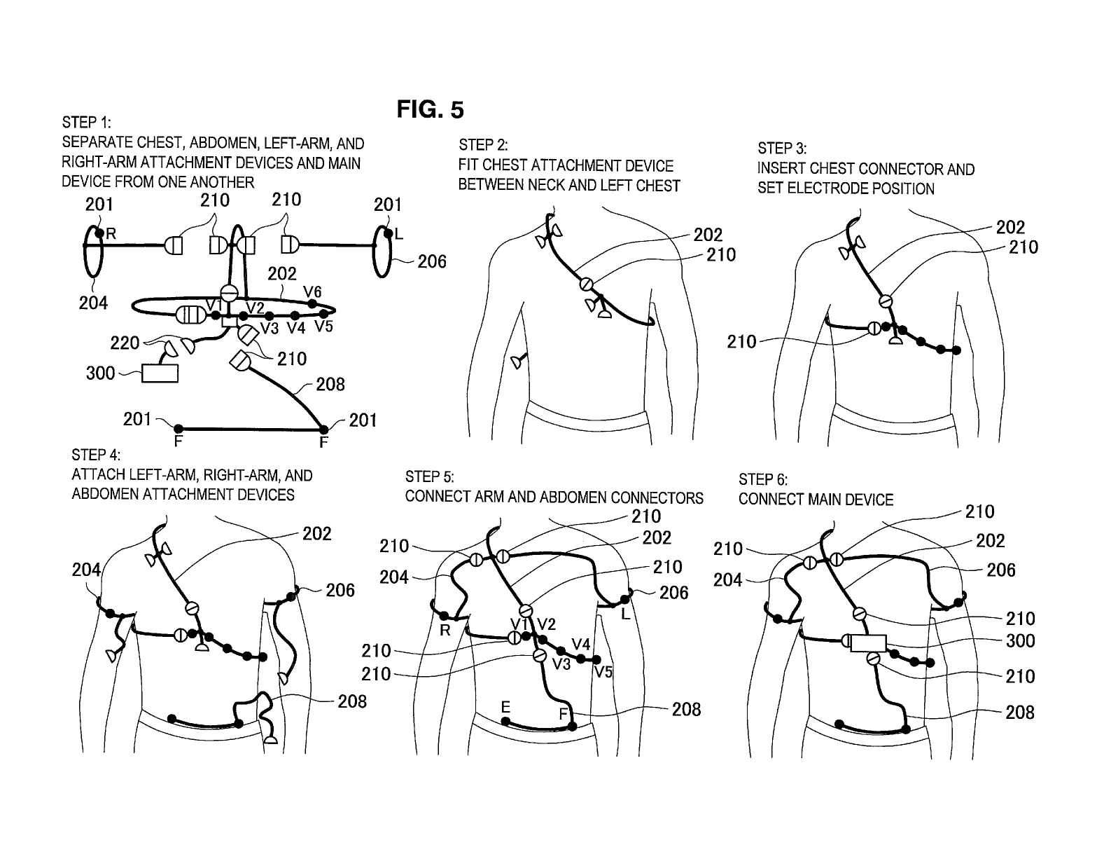

FIG. 5 schematically illustrates a method of how the electrode attachment device 200 is attached. When attaching the electrode attachment device 200, the electrode attachment device 200 is attached to the body in the order of steps 1 to 6 below. The electrode attachment device 200 is detached from the body by reversing the order.

In step 1, the chest attachment device 202, the right-arm attachment device 204, the left-arm attachment device 206, the abdomen attachment device 208, and the main device 300 are separated from one another.

In step 2, the chest attachment device 202 is fitted between the neck and the left chest.

In step 3, one of the connectors 210 is brought entirely around the chest and is connected to the other connector 210, and the electrode position is set to a desired position.

In step 4, the right-arm attachment device 204 is fitted around and attached to the right arm, the left-arm attachment device 206 is fitted around and attached to the left arm, and the abdomen attachment device 208 is attached to the abdomen.

In step 5, the connectors 210 are joined.

In step 6, the assembly connector 220 is joined, and the main device 300 is connected thereto.

1.5. Process for Checking that Electrodes are Properly Attached

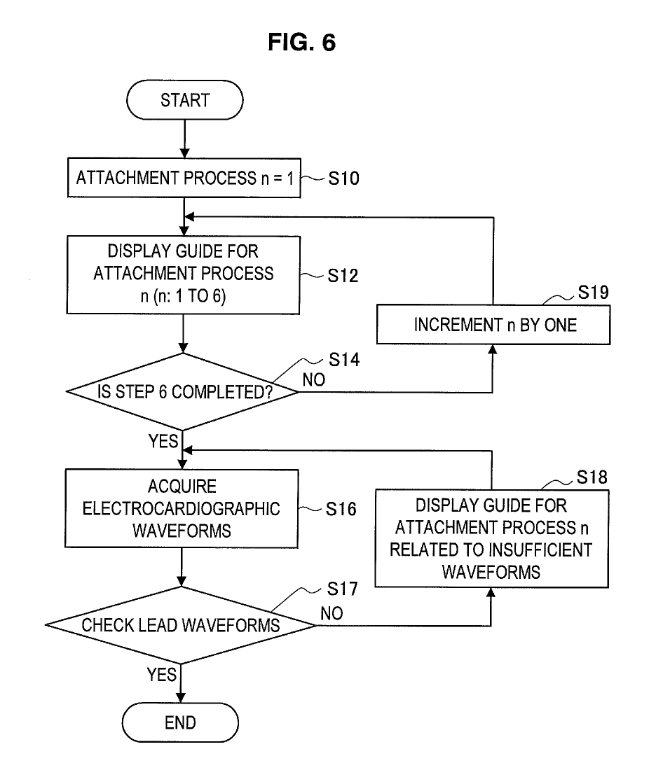

FIG. 6 is a flowchart illustrating a process for checking that the electrodes are properly attached. In step S10, a value of an attachment process n is set to 1 (n=1). In this case, the value n is an integer ranging from 1 to 6, and the process n corresponds to steps 1 to 6 in FIG. 5.

In step S12, a guide for the attaching method in the attachment process n is displayed on the display unit 358 of the electronic apparatus 350. In step S14, it is determined whether or not step 6 is completed. If step 6 is completed, the process proceeds to step S16. If step 6 is not completed, the process proceeds to step S19 where n is incremented by one, and the process returns to step S12.

In step S16, electrocardiographic waveforms are acquired. In step S17, lead waveforms are checked on the basis of the waveforms acquired by the ten electrodes 201. If there are no problems in the lead waveforms, the process ends. If the lead waveforms are insufficient, the process proceeds to step S18 where a guide for an attachment process n related to insufficient waveforms is displayed. After step S18, the process returns to step S16 where the lead waveforms are checked again.

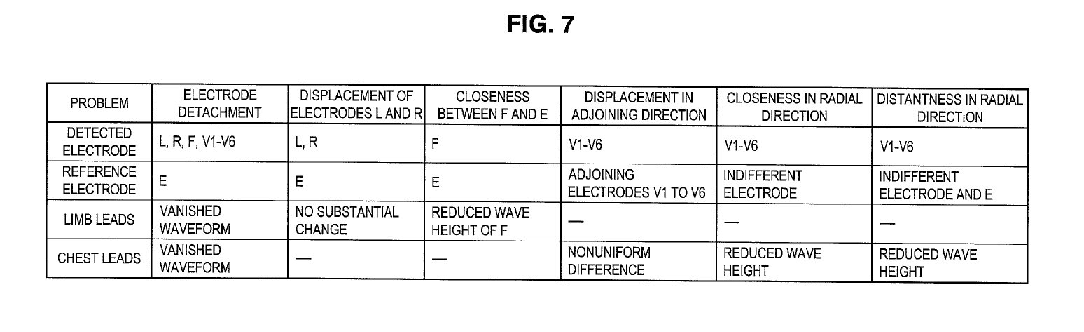

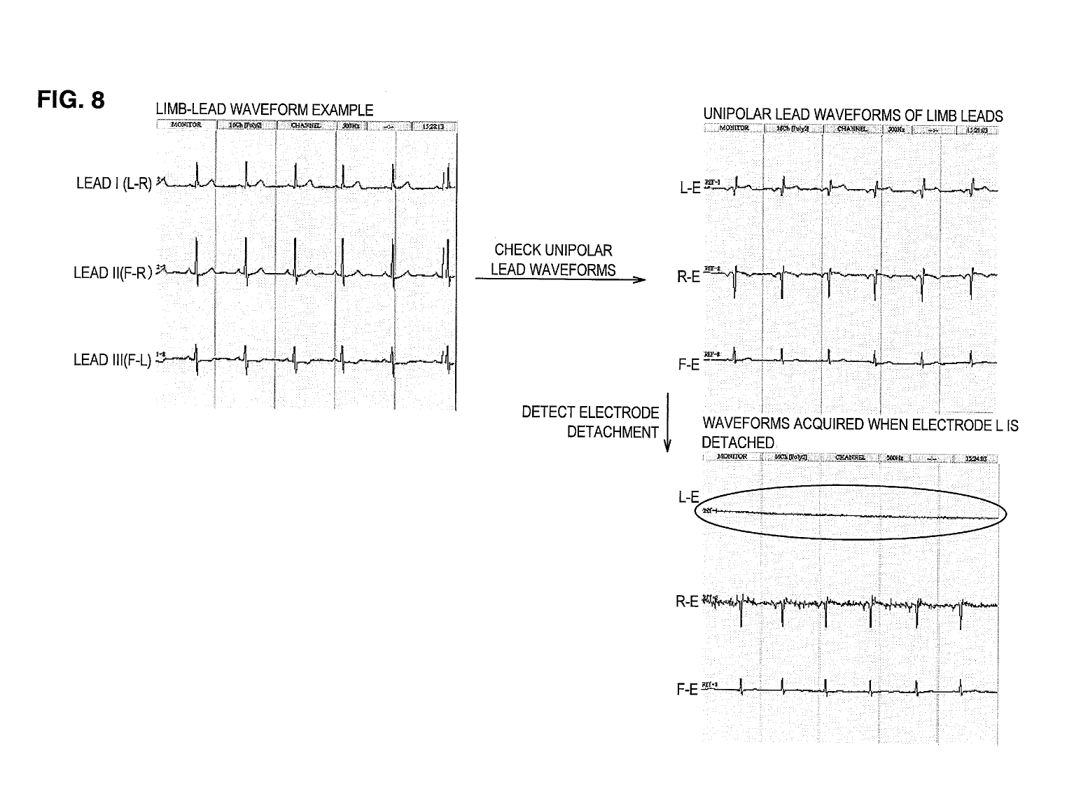

FIG. 7 schematically illustrates a problem detection method based on the lead waveforms. FIGS. 8 to 12 are characteristic diagrams illustrating specific examples of problem detection based on the lead waveforms.

As shown in FIG. 7, detachment of the electrodes L, R, F, and V1 to V6, displacement of the electrodes L and R, closeness between the electrodes F and E, displacement of the electrodes V1 to V6 in the adjoining direction, closeness of the electrodes V1 to V6 in the radial direction, and distantness of the electrodes V1 to V6 in the radial direction can be detected on the basis of the lead waveforms. Specifically, it is possible to detect that the electrodes L, R, F, and V1 to V6 are detached if the waveform of each electrode has vanished with reference to the waveform of the electrode E. It is possible to detect that the electrodes L and R are displaced if there are no substantial changes in the waveform of each electrode with reference to the waveform of the electrode E. It is possible to detect that the electrodes F and E are too close to each other on the basis of reduced wave height of the electrode F with reference to the waveform of the electrode E. It is possible to detect that the electrodes V1 to V6 are displaced in the adjoining direction on the basis of nonuniform differences with reference to adjoining electrodes V1 to V6. It is possible to detect the closeness of the electrodes V1 to V6 in the radial direction on the basis of reduced wave height with reference to an indifferent electrode. It is possible to detect the distantness of the electrodes V1 to V6 in the radial direction on the basis of reduced wave height with reference to an indifferent electrode and the electrode E.

FIG. 8 illustrates waveforms showing that the electrodes L, R, and F are detached. By checking unipolar lead waveforms from limb-lead waveforms, it can be detected that the electrode L is detached if an L-E waveform has vanished. Detachment of the other electrodes R and F can be detected in a similar manner.

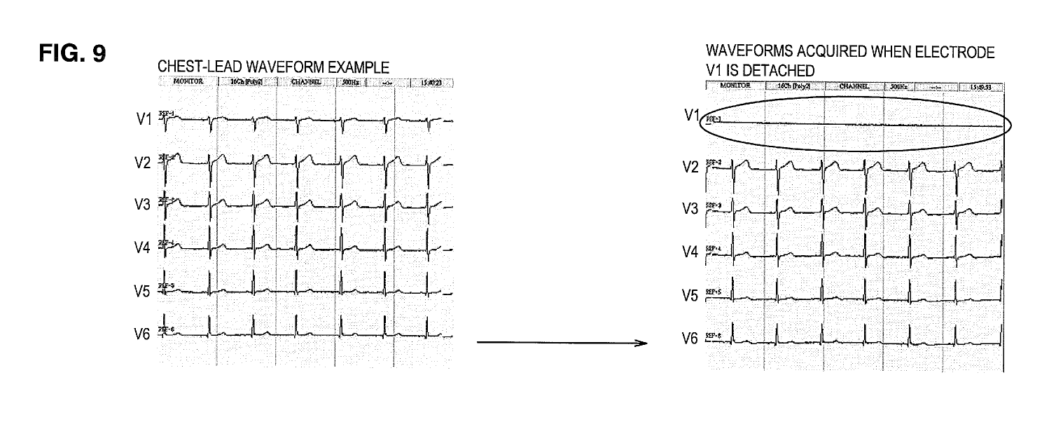

FIG. 9 illustrates waveforms showing that the electrode V1 is detached. If the waveform of the electrode V1 has vanished from chest-lead waveforms, it can be detected that the electrode V1 is detached. Detachment of the electrodes V2 to V6 can be detected in a similar manner.

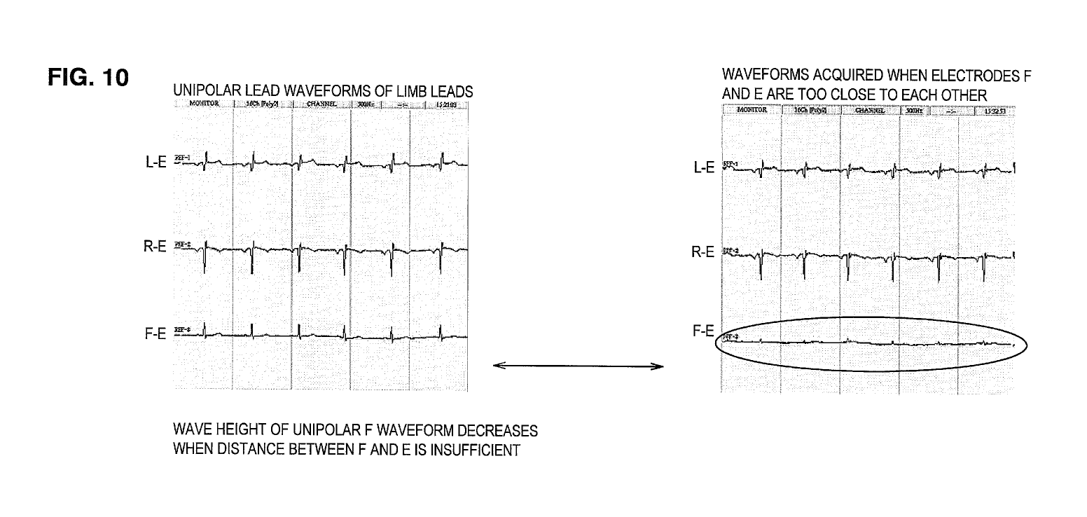

FIG. 10 illustrates waveforms showing that the electrodes F and E are too close to each other. If the distance between the electrodes F and E is insufficient on the basis of unipolar lead waveforms of limb leads, the wave height of the unipolar F waveform decreases. Thus, it can be detected that the electrodes F and E are too close to each other. The detection can be similarly performed for the remaining electrodes.

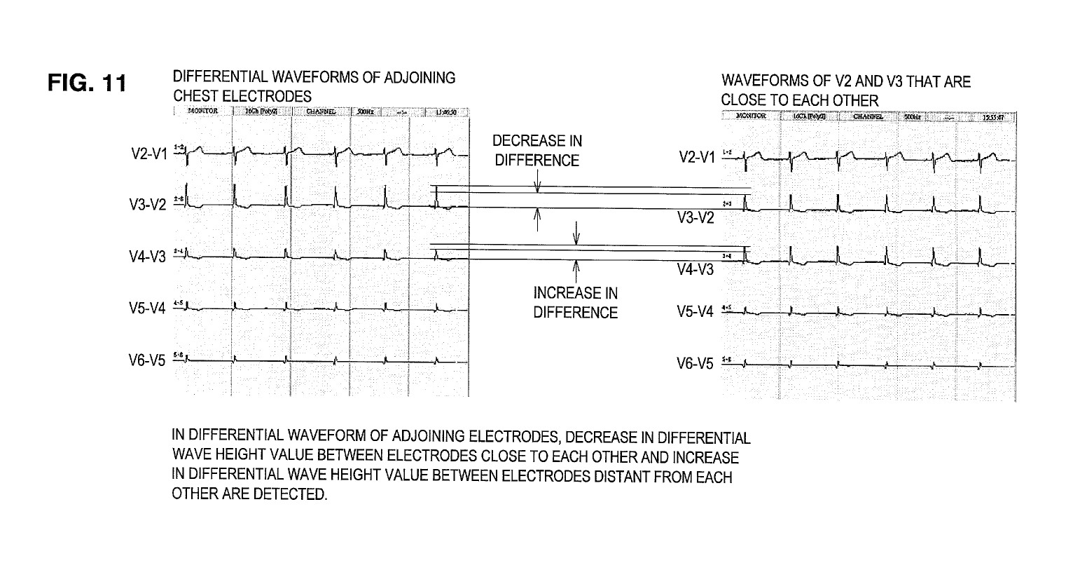

FIG. 11 illustrates waveforms showing that the electrodes V3 and V2 are too close to each other. The closeness between electrodes can be detected based on the fact that, in a differential waveform of adjoining electrodes, a differential wave height value between electrodes that are close to each other decreases and a differential wave height value between electrodes that are distant from each other increases. In the example shown in FIG. 11, the differential waveform of the electrodes V3 and V2 has decreased from the normal, and the differential waveform of the electrodes V4 and V3 has increased from the normal, thereby detecting that the electrodes V3 and V2 are too close to each other. The detection can be similarly performed for the remaining electrodes.

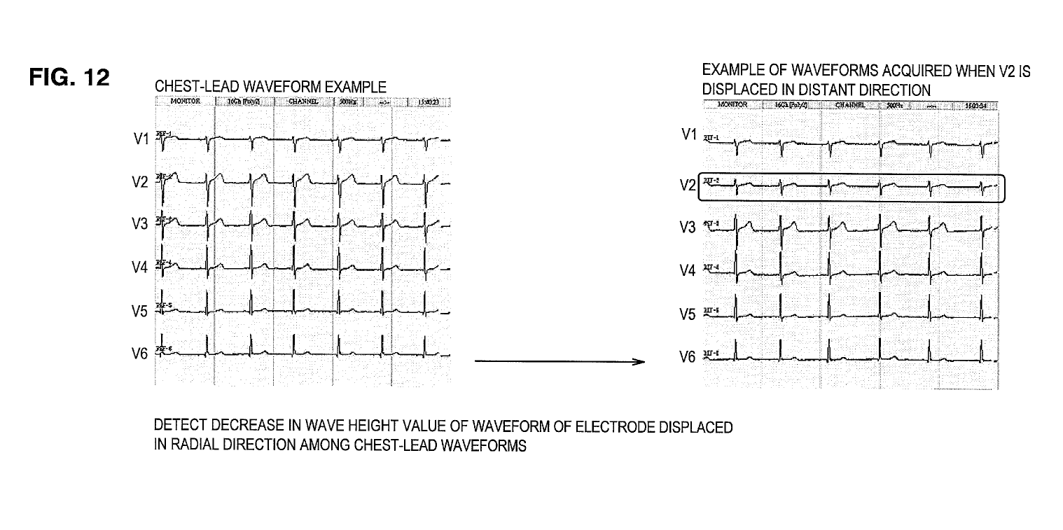

FIG. 12 illustrates waveforms showing distantness of the electrode V2 in the radial direction. In chest-lead waveforms, the wave height value of the waveform of the electrode V2 displaced in the radial direction decreases so that the distantness of the electrode V2 in the radial direction can be detected. The detection can be similarly performed for the remaining electrodes.

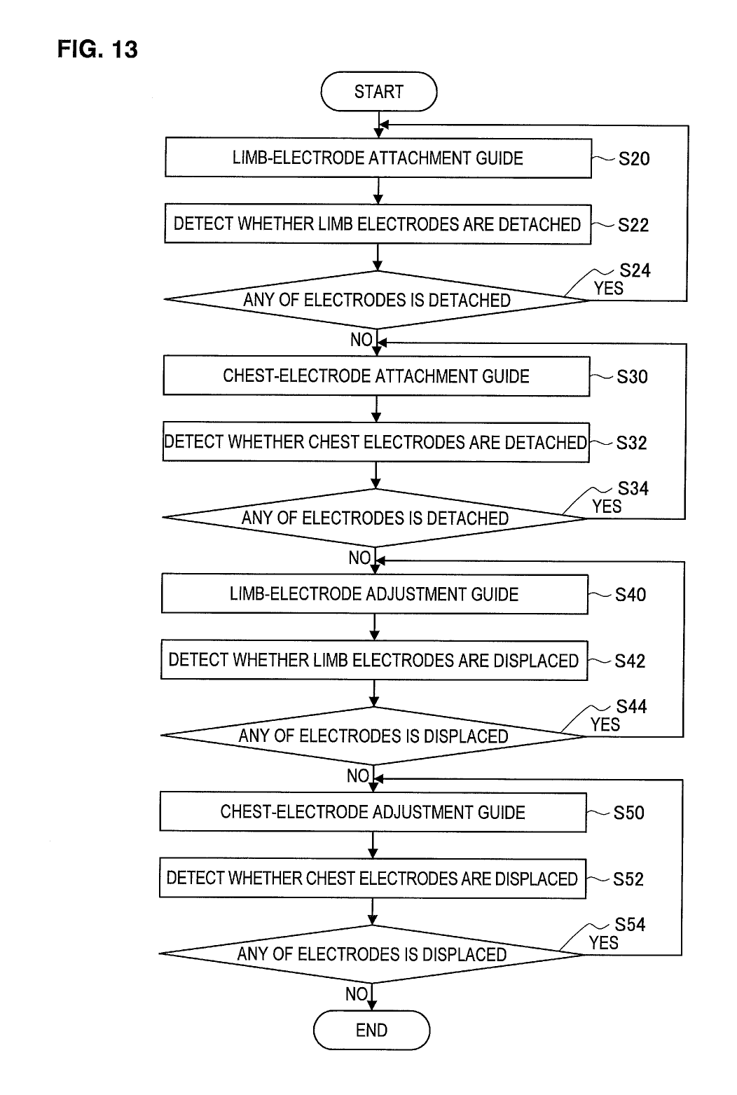

FIG. 13 is a flowchart illustrating a problem detection process based on FIGS. 7 to 12. The flowchart shows the process from steps S16 to S18 in FIG. 6 in detail. First, in step S20, a limb-electrode attachment guide is displayed. Based on the limb-electrode attachment guide, a user attaches the limb electrodes (R, L, F, and E) to himself/herself. In step S22, detection for determining whether the limb electrodes are detached is performed. In step S24, if it is determined that any of the limb electrodes is detached, the process returns to step S20 where the limb-electrode attachment guide is displayed again.

In step S30, a chest-electrode attachment guide is displayed. Based on the chest-electrode attachment guide, the user attaches the chest electrodes (V1, V2, V3, V4, V5, and V6) to himself/herself. In step S32, detection for determining whether the chest electrodes are detached is performed. In step S34, if it is determined that any of the chest electrodes is detached, the process returns to step S30 where the chest-electrode attachment guide is displayed again.

In step S40, a limb-electrode adjustment guide is displayed. The user adjusts the positions of the limb electrodes on the basis of the limb-electrode adjustment guide. In step S42, detection for determining whether the limb electrodes are displaced is performed. In step S44, if it is determined that any of the limb electrodes is displaced, the process returns to step S40 where the limb-electrode adjustment guide is displayed again.

In step S50, a chest-electrode adjustment guide is displayed. The user adjusts the positions of the chest electrodes on the basis of the chest-electrode adjustment guide. In step S52, detection for determining whether the chest electrodes are displaced is performed. In step S54, if it is determined that any of the chest electrodes is displaced, the process returns to step S50 where the chest-electrode adjustment guide is displayed again.

As described above, the guide for the attachment process is displayed on the electronic apparatus 350 (such as an apparatus to which data is to be output, or a related personal computer (PC), tablet terminal, or portable telephone). The attached-state determination unit 354 of the electronic apparatus 350 is provided with a function for checking the attached states by analyzing the received waveforms so as to confirm that the electrodes are properly attached. Thus, the user can attach the electrode attachment device 200 to his/her own body by himself/herself. In addition, it can be confirmed whether or not the electrodes are attached to appropriate positions. Consequently, the user can attach the electrode attachment device 200 to his/her body without receiving help from, for example, a doctor or a nurse.

### 1.6. Example of Application to Observational 18-Lead Electrocardiograph

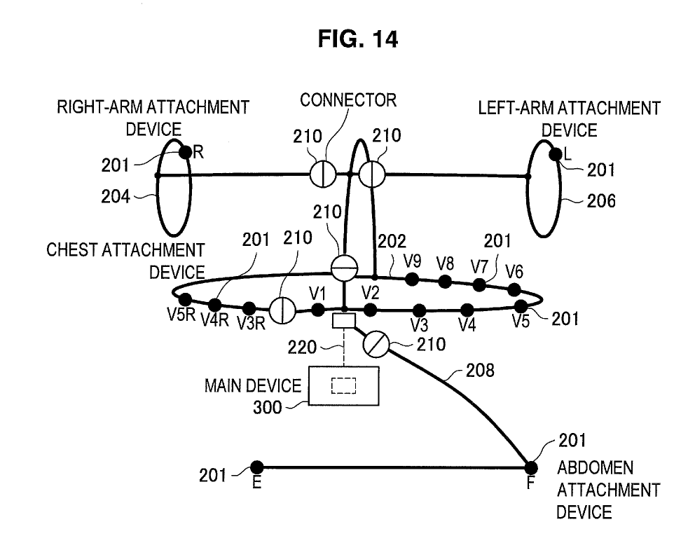

FIG. 14 schematically illustrates the electrode attachment device 200 applied to an observational 18-lead electrocardiograph. In the example shown in FIG. 14, the electrode attachment device 200 shown in FIG. 4 is additionally provided with electrodes V7, V8, V9, V3R, V4R, and V5R. For example, the electrode attachment device 200 can be applied to an observational 18-lead electrocardiograph discussed in JP 4153950B by increasing the number of electrodes in the electrode attachment device 200. Accordingly, without having to make a prediction from a normal 12-lead electrocardiogram, measurement using an observational 18-lead electrocardiograph can be performed in accordance with a process that is the same as the attachment process in the case of a 12-lead electrocardiogram, whereby biological information related particularly to the right ventricle of the heart can be acquired.

According to the first embodiment described above, the electrode attachment device 200 is divided into multiple parts that are connectable by using connectors, so that the user (i.e., patient) can attach the electrode attachment device 200 to his/her body by himself/herself. Furthermore, after attaching the electrode attachment device 200, detachment of the electrodes and positional displacement of the electrodes can be detected on the basis of the lead waveforms. Therefore, the user can acquire, for example, electrocardiographic waveforms by attaching the electrode attachment device 200 to his/her body by himself/herself without being dependent on, for example, a nurse or a helper.

### 2. Second Embodiment

### 2.1. Configuration Example of System According to Second Embodiment

Next, a second embodiment of the present disclosure will be described below. The second embodiment relates to a transmission system in which a biological information sensor (responding apparatus 500) is driven by being supplied with electric power from an information processing apparatus (inquiring apparatus 400) such that the sensor side is passive. Specifically, in this transmission system, the sampling intervals of biological information are maintained with high accuracy.

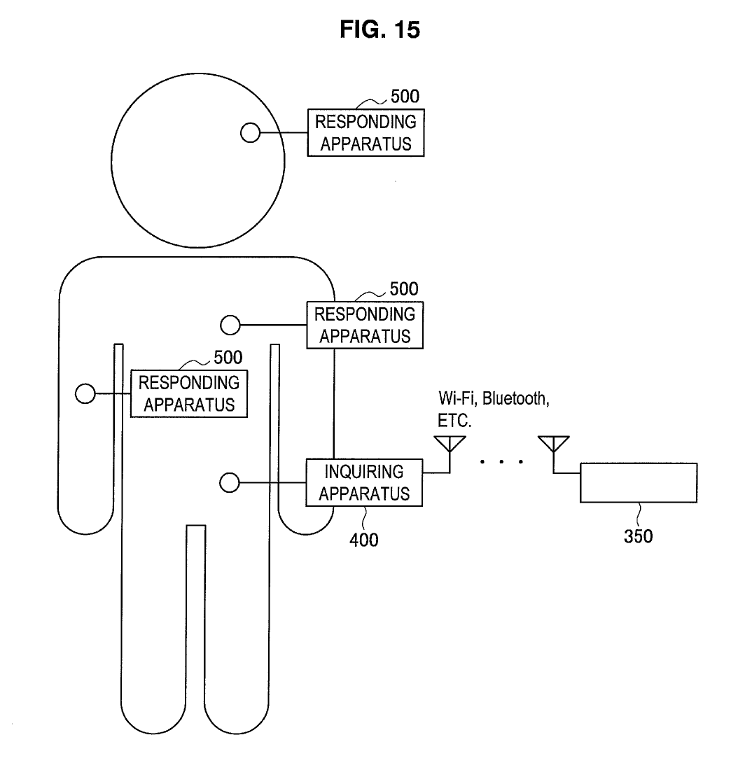

First, the schematic configuration of the system according to the second embodiment of the present disclosure will be described with reference to FIG. 15. The system according to this embodiment includes an inquiring apparatus 400, responding apparatuses 500, and an electronic apparatus 350. The responding apparatuses 500 are configured to be attached to a human body and acquire waveforms as biological information. Therefore, the responding apparatuses 500 are equipped with electrodes that acquire waveforms as biological information. The responding apparatuses 500 correspond to the biological-information acquisition apparatus 100 according to the first embodiment. The inquiring apparatus 400 corresponds to the communication apparatus 330 according to the first embodiment. In the example shown in FIG. 15, one inquiring apparatus 400 and three responding apparatuses 500 are attached to the body.

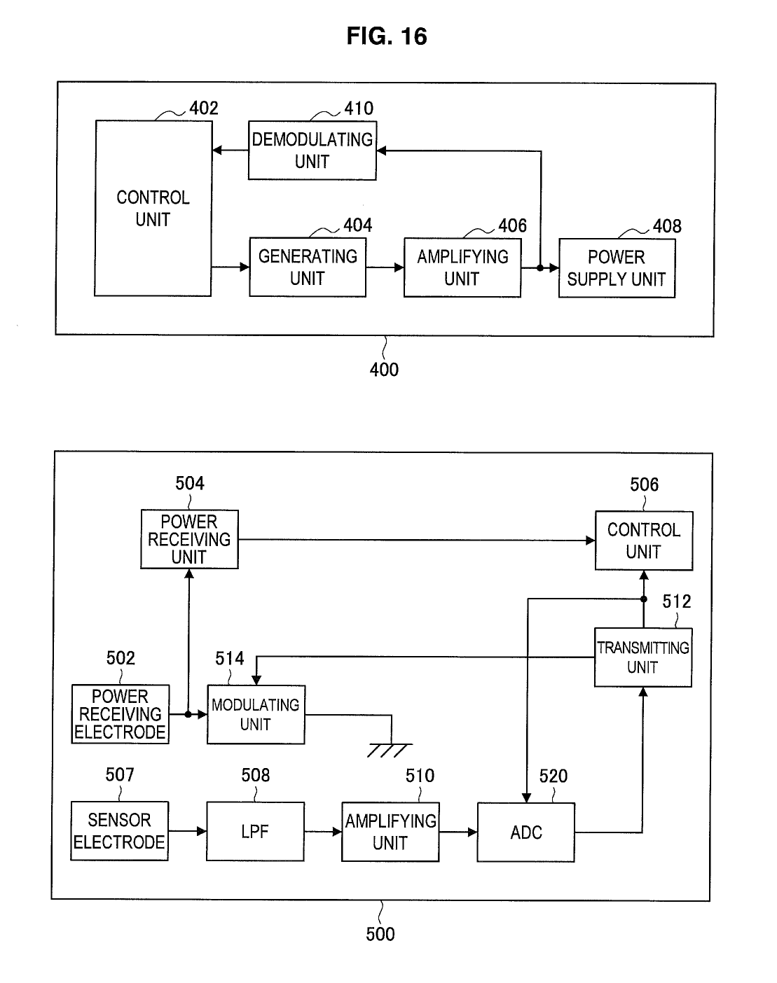

FIG. 16 schematically illustrates the system including the inquiring apparatus 400 and the responding apparatuses 500. Each responding apparatus 500 is equipped with a sensor electrode and acquires biological information from a weak sensor signal. Each responding apparatus 500 activates a transmission circuit only when the responding apparatus 500 receives an inquiry signal with a specific oscillation frequency. In other words, the responding apparatus 500 does not activate its own transmission circuit when another responding apparatus 500 activates its transmission circuit, so that undesired noise is not generated in other periods in which the responding apparatuses 500 do not act as undesired noise generating sources against each other. Thus, each responding apparatus 500 can acquire a biological signal from a weak sensor signal without being affected by noise from another responding apparatus 500.

The inquiring apparatus 400 includes a control unit 402 that controls the overall operation of the inquiring apparatus 400, a generating unit 404 that generates an alternating current signal for electric power supply, an amplifying unit 406 that amplifies the alternating current signal generated by the generating unit 404, a power supply unit 408 that sends out the amplified alternating current signal, and a demodulating unit 410 that receives a response signal from each responding apparatus 500 and demodulates the response signal so as to acquire biological information data.

The control unit 402 controls the overall operation of the inquiring apparatus 400 in addition to causing the inquiring apparatus 400 to exchange information with external apparatuses, such as the responding apparatuses 500. The generating unit 404 has an oscillation-frequency changing function and generates an alternating current signal with a specific frequency in accordance with a command from the control unit 402. The term “specific frequency” in this case refers to a resonant frequency with which a reception circuit of each responding apparatus 500 synchronizes. The alternating current signal output from the generating unit 404 is appropriately amplified by the amplifying unit 406 and is subsequently supplied to the power supply unit 408. The power supply unit 408 is in contact with the human body acting as a communication medium, such as a hand. The supplied alternating current signal is sent out to the human body as an inquiry signal constituted of an unmodulated carrier wave so as to reach each responding apparatus 500.

Any one of the responding apparatuses 500 having a reception circuit that synchronizes with the frequency of the unmodulated carrier wave transmitted from the inquiring apparatus 400 generates electric power from the received unmodulated carrier wave and then utilizes this electric power to activate the transmission circuit. Then, the transmission circuit generates a response signal by superimposing information (e.g., biological information such as the heart rate) onto this unmodulated carrier wave, and transmits the response signal via the human body acting as a medium.

When the power supply unit 408 receives the aforementioned response signal, the inquiring apparatus 400 uses the demodulating unit 410 to extract the information superimposed on the response signal. When the control unit 402 determines that the information, such as the biological information, is completely acquired from one of the responding apparatuses 500, the control unit 402 subsequently commands the generating unit 404 to change the oscillation frequency so as to acquire information from another responding apparatus 500. Then, an inquiry signal constituted of an unmodulated carrier wave with a different frequency is sequentially transmitted from the power supply unit 408 via the human body acting as a medium.

Each responding apparatus 500 includes a power receiving electrode 502 that receives an alternating current signal from the inquiring apparatus 400 so as to acquire biological information, a power receiving unit 504 having a resonant circuit that resonates at a frequency specific to each responding apparatus 500, a control unit 506 that controls the overall operation including, for example, requesting acquisition of biological information and generation of a response signal after receiving electric power, a low-pass filter (LPF) 508 that acquires biological information in a desired band from a signal obtained from a sensor electrode 507, an amplifying unit 510 that amplifies the filtered biological information, an analog-to-digital conversion circuit (ADC) 520, a transmitting unit 512 that generates a biological information data string to be transmitted, and a modulating unit 514 that generates a transmission signal by performing modulation on the received unmodulated carrier on the basis of the biological information data.

The power receiving electrode 502 is in contact with a predetermined part of the human body acting as a communication medium. The unmodulated carrier wave with the specific frequency transmitted from the inquiring apparatus 400 via the human body acting as a medium can be received by the power receiving electrode 502.

The power receiving unit 504 is equipped with a resonant circuit (not shown) that resonates at a frequency specific to the responding apparatus 500 relative to the signal received by the power receiving electrode 502. Furthermore, the power receiving unit 504 is configured to generate electric power with constant voltage from an output from this resonant circuit, detect whether the reception voltage is sufficient for driving the responding apparatus 500, and output a power-supply detection signal. Since the responding apparatus 500 is capable of returning a response signal only when it receives the specific frequency, the unmodulated carrier wave with the specific frequency serves as an inquiry signal.

The control unit 506 controls the operation of the entire responding apparatus 500. When the control unit 506 receives the power-supply detection signal from the power receiving unit 504, the control unit 506 sends a command for acquisition of biological information and transmission of a response signal having the acquired biological information superimposed thereon.

The sensor electrode 507 is in contact with a predetermined part of the human body and detects, for example, the heart rate so as to output a sensor signal. With regard to the sensor signal, a component thereof in a desired band is extracted (i.e., an undesired component thereof is removed) by the low-pass filter 508 and is appropriately amplified by the amplifying unit 510. Moreover, the component is sampled and quantized by the ADC 520 so as to become digital biological information.

When the transmitting unit 512 receives a command for transmission of a response signal from the control unit 506, the transmitting unit 512 digitally modulates the biological information acquired from the ADC 520 in accordance with a predetermined format. The modulating unit 514 performs modulation on the unmodulated carrier wave received by the power receiving electrode 502 on the basis of the digitally modulated transmission information. The modulated carrier wave is sent out as a response signal from the power receiving electrode 502 to the human body acting as a communication medium.

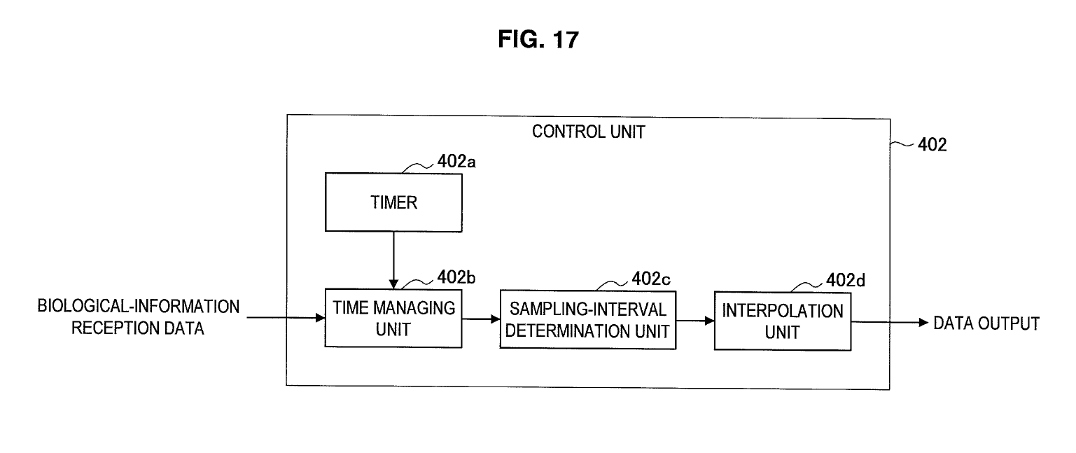

FIG. 17 is a schematic functional block diagram of the control unit 402 of the inquiring apparatus 400. The control unit 402 includes a timer 402a, a time managing unit 402b, a sampling-interval determination unit 402c, and an interpolation unit 402d. The timer 402a is provided for acquiring biological information data at constant sampling intervals and provides a timeout notification at constant time intervals. The time managing unit 402b counts a time interval from a time point at which timeout is notified by the timer 402a to a time point at which reception data is acquired. The sampling-interval determination unit 402c compares a time interval set on the basis of pre-designed operation with a time interval taken to acquire the reception data this time. If the sampling-interval determination unit 402c determines that there is a deviation in a sampling interval, the corresponding data is deleted. Furthermore, in the second embodiment, if the sampling-interval determination unit 402c determines that there is a deviation in the sampling interval, it is more preferable that data that would have been received at a desired sampling interval be interpolated at the interpolation unit 402d. If the sampling-interval determination unit 402c determines that there is no deviation in the sampling interval, the reception data is directly output.

The biological information is of various kinds, such as a body temperature, pulse, respiration, blood pressure, SpO₂, an electrocardiogram, an electromyogram, brain waves, or body motion. Depending on the kind of biological information, high accuracy may be demanded for the sampling interval, or the accuracy of the sampling interval may be relatively low. For example, since body temperature is not information that fluctuates rapidly, an effect is relatively low even if the sampling interval for the information deviates by, for example, several milliseconds. However, with regard to biological information that has a major significance on the shape of waveforms, such as an electrocardiogram, the biological information loses its medical value if the sampling intervals fluctuate.

In the system configuration constituted of the inquiring apparatus 400 serving as an information processing apparatus and the multiple responding apparatuses 500 serving as biological information sensors, as shown in FIG. 15, in order to perform data communication smoothly between the inquiring apparatus 400 and each responding apparatus 500, the system is designed such that the multiple responding apparatuses 500 do not respond simultaneously. Therefore, for example, each responding apparatus 500 has a reception circuit for decoding (i.e., comprehending) a request (i.e., an inquiry) from the inquiring apparatus 400 and continues to wait until a request is transmitted from the inquiring apparatus 400. Thus, the size of the apparatus and the power consumption thereof tend to increase.

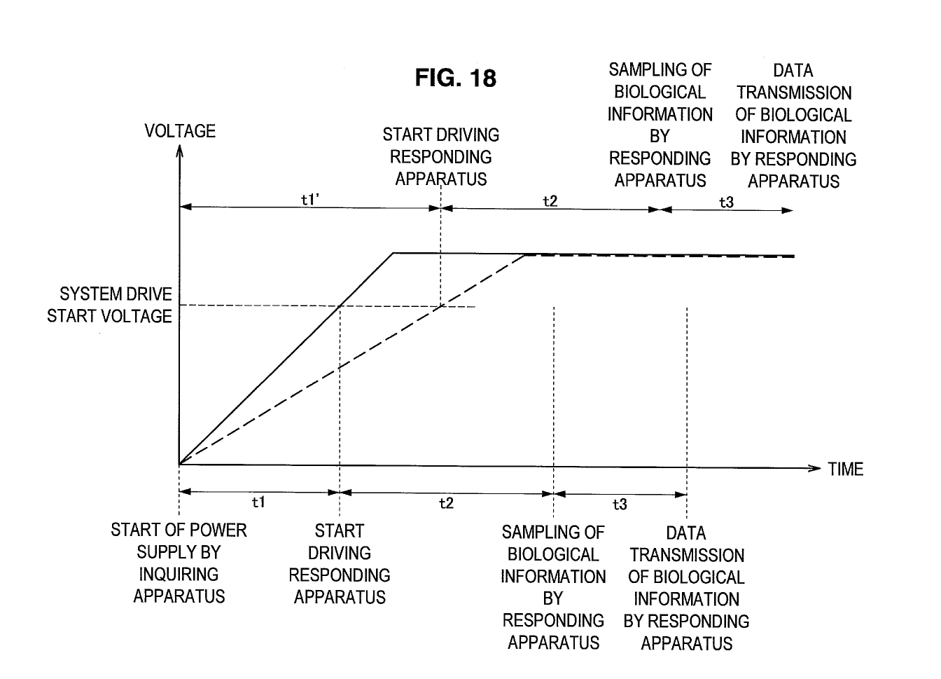

As described above, in JP 2012-235565A, each responding apparatus is driven by being supplied with electric power from the inquiring apparatus. The time that it takes to start driving the responding apparatus 500 varies depending on the condition of resonance between the inquiring apparatus and the responding apparatus. FIG. 18 is a characteristic diagram expressing the relationship between reception voltage and time in the power receiving unit 504 of the responding apparatus 500. The time it takes to reach sufficient reception voltage in the power receiving unit 504 of the responding apparatus 500 after the inquiring apparatus 400 starts supplying electric power thereto is defined as t1. The time it takes to start sampling the biological information after starting the driving of the responding apparatus 500 is defined as t2. The time it takes to start transmitting information to the inquiring apparatus 400 after starting the sampling of the biological information is defined as t3. Each of t2 and t3 is the time it takes to perform pre-designed operation and is a characteristic value. On the other hand, t1 may possibly be affected by, for example, the distance or the positional relationship between the inquiring apparatus 400 and the responding apparatus 500 and may thus change to t1′. When t1 changes to t1′, the drive start timing for the responding apparatus 500 becomes delayed, thus causing a delay in the sampling of the biological information and the data transmission of the biological information. This may result in a difficulty in keeping the sampling intervals of the biological information constant.

### 2.2. Operation Sequence of Inquiring Apparatus and Responding Apparatus

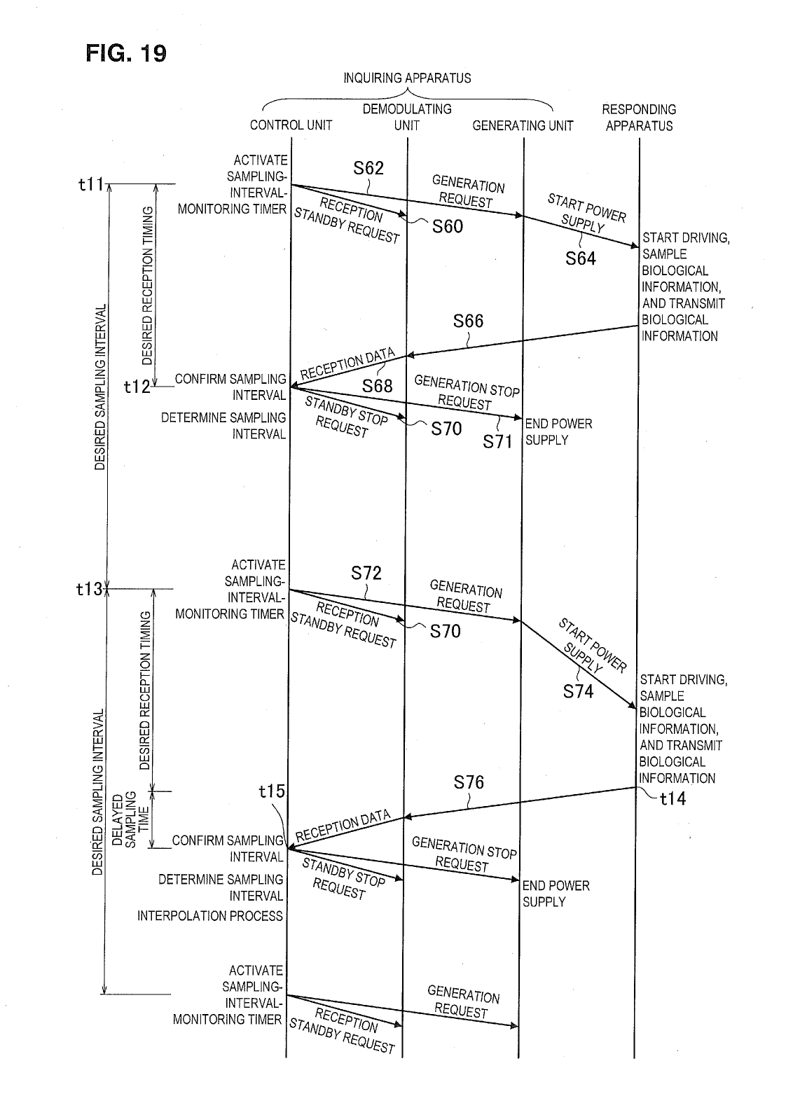

FIG. 19 is a sequence diagram illustrating the operation of the inquiring apparatus 400 and each responding apparatus 500. In step S60, the control unit 402 activates the timer 402a, which is configured to monitor the sampling intervals, at a time point t11 and sends a reception standby request to the demodulating unit 410. In step S62, the control unit 402 sends a generation request to the generating unit 404. In step S64, the generating unit 404 receives the generation request and starts supplying electric power to the responding apparatus 500. In step S66, the responding apparatus 500 receives the supplied electric power, and the driving thereof commences when the voltages reaches a drive start voltage. Then, the responding apparatus 500 samples the biological information and transmits the biological information.

In step S68, the demodulating unit 410 of the inquiring apparatus 400 receives the biological information and transmits the demodulated reception data to the control unit 402. When the control unit 402 receives the biological information at a time point t12, a sampling interval is confirmed. Then, the control unit 402 performs a determination process with respect to the sampling interval.

In step S70, the control unit 402 sends a standby stop request to the demodulating unit 410. In step S71, the control unit 402 sends a generation stop request to the generating unit 404. Consequently, the supply of electric power to the responding apparatus 500 stops.

Subsequently, in step S70, the control unit 402 activates the sampling-interval-monitoring timer 402a at a time point t13 and sends a reception standby request to the demodulating unit 410. In step S72, the control unit 402 sends a generation request to the generating unit 404. In step S74, the generating unit 404 receives the generation request and starts supplying electric power to the responding apparatus 500. Although the driving of the responding apparatus 500 commences when the received electric power reaches the drive start voltage, the time it takes to start driving the responding apparatus 500 after commencing the supply of electric power thereto in step S74 is delayed as compared with step S64. Therefore, a time point t14 at which the responding apparatus 500 samples the biological information and transmits the biological information in step S76 is delayed. As a result, a time point t15 at which the control unit 402 receives the reception data transmitted from the demodulating unit 410 receiving the biological information is also delayed.

### 2.3. Interpolation Process by Interpolation Unit

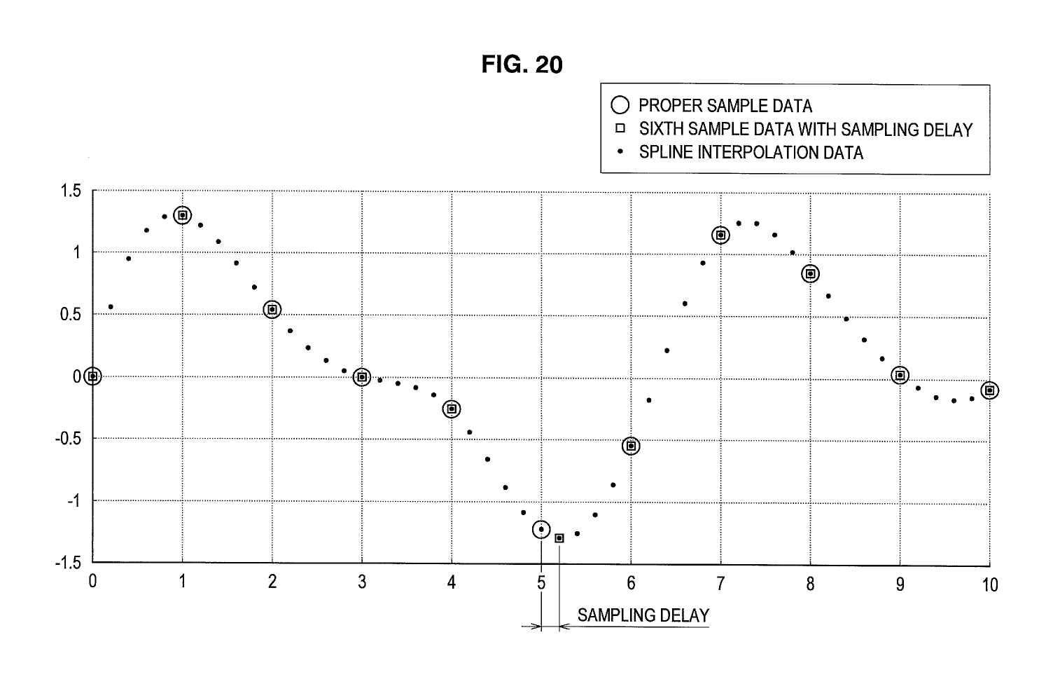

Due to the above reason, the interpolation unit 402d of the control unit 402 interpolates data that would have been received at the desired sampling interval. FIG. 20 illustrates an interpolation process performed in the interpolation unit 402d and is a characteristic diagram showing time-series data of biological information in a certain period. In FIG. 20, circles denote data properly sampled at constant intervals. Squares in FIG. 20 denote actually sampled data. As shown in FIG. 20, sixth sample data is sampled at a time point that is slightly delayed from a time point t=5. The interpolation unit 402d generates interpolation data between samples by using these sample data and sampling intervals. Small black circles in FIG. 20 denote data interpolated by spline interpolation. Accordingly, by spline interpolation, interpolation data (small black circle) at the time point t=5 is aligned with properly sampled data (circle).

Accordingly, in the second embodiment, the inquiring apparatus 400 serving as an information processing apparatus manages time and monitors fluctuations in the sampling intervals of the biological information by calculating the sampling time from the electric-power-supply start timing for the responding apparatus 500 and the timing at which biological information data is received from the responding apparatus 500. The inquiring apparatus 400 discards biological information data if received at a time point that is deviated from a desired sampling interval and uses a biological information data string only constituted of highly reliable sample data as data of medical value. Furthermore, if there is a deviation from a desired sampling interval, the inquiring apparatus 400 interpolates data corresponding to a desired sampling time point in accordance with sample data obtained before and after the sample data corresponding to the deviation. Thus, in a transmission system in which a biological information sensor is driven by being supplied with electric power from an information processing apparatus such that the sensor side is passive, the system can handle biological information in which accurate sampling intervals are demanded.

According to the second embodiment described above, in a transmission system in which a biological information sensor (i.e., responding apparatus 500) is driven by being supplied with electric power from an information processing apparatus (inquiring apparatus 400) such that the sensor side is passive, the information processing apparatus manages time and calculates the sampling time from the electric-power-supply start timing for the biological information sensor and the timing at which biological information data is received from the biological information sensor. If there is a deviation in a sampling interval, the information processing apparatus interpolates data corresponding to a desired time point. Consequently, the system can handle biological information in which accurate sampling intervals are demanded.

### 3. Third Embodiment

### 3.1. Configuration Example of Electrode According to Third Embodiment

Next, a third embodiment of the present disclosure will be described below. The third embodiment relates to the configuration of each electrode in the electrode attachment device 200 according to the first embodiment. Although each electrode is to be attached directly to the body, the electrode may easily detach from the body if the adhesive force of the electrode is weak, making it difficult to acquire biological information stably. On the other hand, a strong adhesive force of the electrode makes it difficult to detach the electrode from the body.

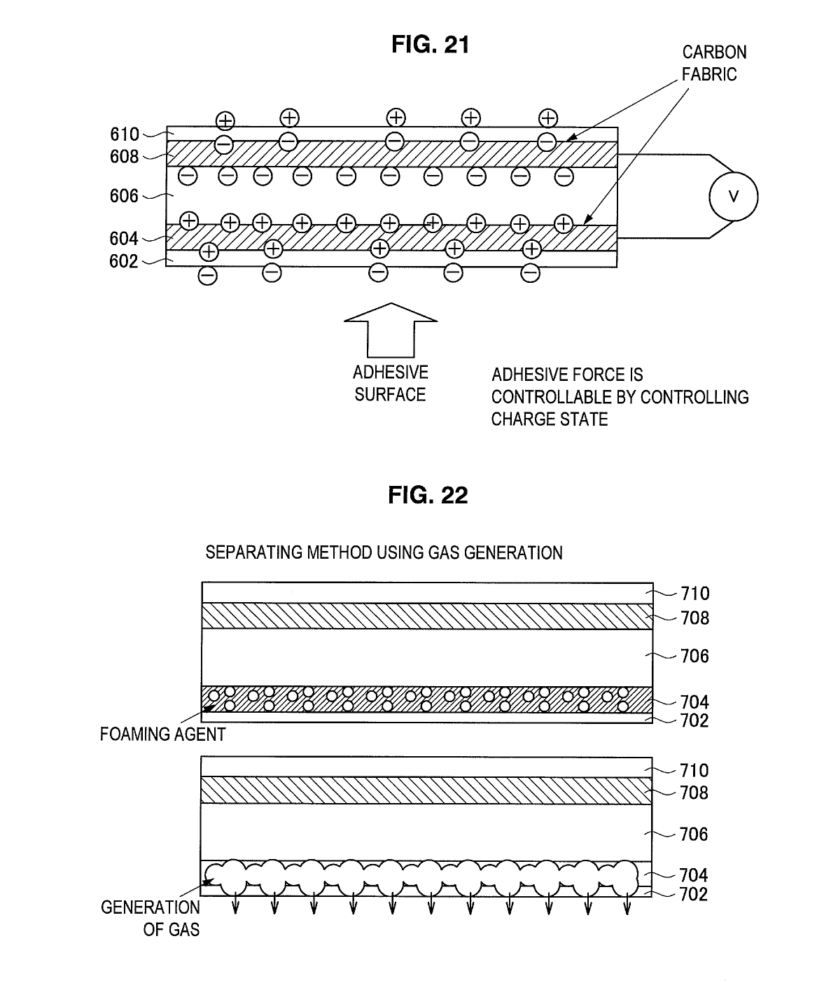

The third embodiment provides a structure that allows for reliable attachment of each electrode to the body by increasing the adhesive force of the electrode to the body and that also allows for easy detachment of the electrode from the body. Electrodes 600 and 700 to be described below with reference to FIGS. 21 and 22 correspond to the electrodes 201 according to the first embodiment. FIG. 21 is a schematic cross-sectional view illustrating the configuration of the electrode 600 according to the third embodiment. As shown in FIG. 21, the electrode 600 is formed by laminating an adhesive layer 602, a carbon fiber layer 604, an electrolyte layer 606, a carbon fiber layer 608, and an adhesive layer (insulating layer) 610 in this order from below. The carbon fiber layers 604 and 608 are each formed of carbon fiber fabric. The electrolyte layer 606 and the adhesive layers 602 and 610 are each composed of a material having adhesive force and high ionic conductivity. For example, the electrolyte layer 606 and the adhesive layers 602 and 610 are each composed of a polyethylene-ethylene-oxide-hexamethylene copolymer or styrene-butadiene-rubber (SBR) polyethylene-oxide copolymer impregnated with an ionic material and are disposed so as not to conduct electricity to the carbon fiber layers 604 and 608.

In FIG. 21, the adhesive layer 602, which is the lower layer, is adhered to the body of the user. The carbon fiber layer 604 and the carbon fiber layer 608 each receive a predetermined potential. In the state where the adhesive layer 602 is adhered to the body, no potential difference is applied between the carbon fiber layer 604 and the carbon fiber layer 608. On the other hand, when the electrode 600 is to be detached from the body by separating the adhesive layer 602 off from the body, a predetermined potential difference is applied between the carbon fiber layer 604 and the carbon fiber layer 608.

The body of the user is hydrophilic, whereas the adhesive layer 602 is hydrophobic. The adhesive layer 602 is adhered to the body owing to a difference in surface tension between the adhesive layer 602 and the body. In this state, when a predetermined potential difference is applied between the carbon fiber layer 604 and the carbon fiber layer 608, negative charge is generated over the surface of the adhesive layer 602, thus causing the adhesive layer 602 to become hydrophilic. Thus, the difference in surface tension between the adhesive layer 602 and the body decreases, whereby the adhesive force of the adhesive layer 602 to the body decreases. Consequently, by producing a predetermined potential difference between the carbon fiber layer 604 and the carbon fiber layer 608, the electrode 600 becomes readily detachable from the body. Accordingly, by applying voltage between the two carbon fiber layers 604 and 608, the adhesive force can be controlled.

Therefore, even with the sufficiently increased adhesive force of the adhesive layer 602 to the body, the electrode 600 can be readily detached from the body by applying voltage between the two carbon fiber layers 604 and 608 when detaching the electrode 600 from the body. With the configuration shown in FIG. 21, the electrode 600 can be readily separated off from the human body without adversely affecting the body even with the use of the adhesive layer 602 having high adhesive force.

FIG. 22 is a schematic cross-sectional view illustrating another example of an electrode according to this embodiment. As shown in FIG. 22, the electrode 700 is formed by laminating an adhesive layer 702, a carbon fiber layer 704, an electrolyte layer 706, a carbon fiber layer 708, and an adhesive layer (insulating layer) 710 in this order from below. The carbon fiber layers 704 and 708 are each formed of carbon fiber fabric. With regard to the carbon fiber layer 704 located at the adhesive layer 702 side, fine particles of a foamable solid material, such as sodium acid carbonate, are mixed in the carbon fiber fabric. The electrolyte layer 706 and the adhesive layers 702 and 710 are similar to the electrolyte layer 606 and the adhesive layers 602 and 610 shown in FIG. 21.

In FIG. 22, the adhesive layer 702, which is the lower layer, is adhered to the body of the user. The carbon fiber layer 704 and the carbon fiber layer 708 each receive a predetermined potential. In the state where the adhesive layer 702 is adhered to the body, no potential difference is applied between the carbon fiber layer 704 and the carbon fiber layer 708. On the other hand, when the electrode 700 is to be detached from the body by separating the adhesive layer 702 off from the body, a predetermined potential difference is applied between the carbon fiber layer 704 and the carbon fiber layer 708.

When a predetermined potential difference is applied between the carbon fiber layer 704 and the carbon fiber layer 708, the foamable solid material contained in the carbon fiber layer 704 foams by reacting to the voltage. Thus, gas is generated from the carbon fiber layer 704 toward the adhesive layer 702. This generated gas reduces the adhesive force of the adhesive layer 702, thus facilitating the detachment of the electrode 700 from the body. Consequently, with the configuration shown in FIG. 22, the electrode 700 can be readily separated off from the human body without adversely affecting the body even with the use of the adhesive layer 702 having high adhesive force.

### 3.2. Method for Manufacturing Electrolyte Layer

Next, a method for manufacturing a polyethylene-oxide-hexamethylene copolymer used for each of the electrolyte layers 606 and 706 shown in FIGS. 21 and 22 will be described. Polyethylene glycol (PEG) 1000 (42 parts by mass), trimethylol propane (42 parts by mass), and hexamethylene diisocyanate (16 parts by mass) are mixed together at a temperature ranging between 50° C. and 60° C., and the mixture in a liquid state undergoes nitrogen bubbling. After performing the bubbling for three or more minutes, the mixture is sealed and is preliminarily polymerized for three hours. The preliminarily polymerized mixture is set in a mold and undergoes polymerization at 60° C. for 20 hours.

Upon completion of this polymer, carbon fabric is set, and a similar preliminary polymer is appropriately added. Then, the polymer undergoes polymerization at 60° C. for another 20 hours. By immersing this polymer into a liquid containing an electrically conductive component, the polymer can be given high conductivity. In a case where the hexamethylene diisocyanate is smaller than or equal to 10 parts by mass, it is difficult to obtain a solid polymer. On the other hand, in a case where the hexamethylene diisocyanate is larger than or equal to 30 parts by mass, the resultant polymer has no flexibility and is not suitable for attachment to the body.

The conductivity of the polyethylene-oxide-hexamethylene copolymer manufactured in the above-described manner is about twice as high as that of an SBR-polyethylene-oxide copolymer, and is thus suitable as a material used for the electrodes 600 and 700. Therefore, the use of polyethylene-oxide-hexamethylene copolymer for forming the electrodes 600 and 700 improves the characteristics of the electrodes 600 and 700 and also facilitates detachment from the body.

With regard to the structure of each electrode, an electrode discussed in any of the following publications applied by the present applicant may be used. The publications include JP2012-239696A (gel elastic electrode), JP2012-110535A (spiral pin electrode), JP2012-5777 (swab-like electrode), and JP2011-140711A (brush-like electrode).

According to the third embodiment described above, the adhesive force of the electrodes 600 and 700 to the body can be increased, and the electrodes 600 and 700 can be readily detached from the body when detaching them therefrom. Consequently, an electrode that allows for reliable acquisition of biological information and that can be readily detached from the body can be provided.

Although preferred embodiments of the present disclosure have been described above in detail with reference to the appended drawings, the technical scope of the present disclosure is not limited to these examples. It should be understood by those having a general knowledge of the technical field of the present disclosure that various modifications and alterations may occur within the technical scope of the appended claims or the equivalents thereof, and such modifications and alterations are included in the technical scope of the present disclosure.

Additionally, the present disclosure may also be configured as below.

(1) A biological-information acquisition apparatus including:

a plurality of flexible attachment devices each provided with an electrode that is attached to a body and that is configured to acquire biological information; and

a connector configured to connect the plurality of attachment devices.

(2) The biological-information acquisition apparatus according to (1), wherein one of the attachment devices is attached to a chest area and acquires an electrocardiographic chest-lead waveform as the biological information.
(3) The biological-information acquisition apparatus according to (1), wherein one of the attachment devices is attached to a right arm or a left arm and acquires an electrocardiographic limb-lead waveform as the biological information.
(4) The biological-information acquisition apparatus according to (1), wherein one of the attachment devices is attached to a hip and acquires an electrocardiographic limb-lead waveform as the biological information.
(5) The biological-information acquisition apparatus according to (1), further including:

a main device configured to acquire the biological information from each of the attachment devices and transmit the biological information to a communication apparatus via intra-body communication.

(6) The biological-information acquisition apparatus according to (5), wherein the main device is connected to one of the attachment devices via the connector.
(7) The biological-information acquisition apparatus according to (5), wherein the communication apparatus transmits the biological information to an electronic apparatus configured to determine whether each electrode is in an attached state based on the biological information.
(8) The biological-information acquisition apparatus according to (7), wherein the electronic apparatus includes a display unit configured to display a guide for attaching the attachment devices to the body.
(9) The biological-information acquisition apparatus according to (1), wherein each electrode is formed by laminating, an adhesive layer attachable to the body, a first conductive layer, an electrolyte layer, and a second conductive layer in this order, and a predetermined potential difference is applied between the first conductive layer and the second conductive layer when the electrode is to be detached from the body.
(10) The biological-information acquisition apparatus according to (9), wherein the electrolyte layer and the adhesive layer are each composed of a polyethylene-ethylene-oxide-hexamethylene copolymer or SBR polyethylene-oxide copolymer impregnated with an ionic material.
(11) The biological-information acquisition apparatus according to (9), wherein the first conductive layer and the second conductive layer are each formed of a carbon fiber layer.
(12) The biological-information acquisition apparatus according to (9), wherein the first conductive layer has a foamable solid material mixed therein.
(13) A communication system including:

a biological-information acquisition apparatus including an electrode that is attached to a body and that is configured to acquire biological information, a transmitting unit configured to transmit the biological information acquired by the electrode, and a power receiving unit configured to receive supplied electric power; and

an information processing apparatus including a power supply unit configured to perform power supply to the biological-information acquisition apparatus via intra-body communication, a receiving unit configured to receive the biological information from the transmitting unit via intra-body communication, a sampling-interval determination unit configured to determine a sampling interval extending from when the power supply commences to when the biological information is received, and an interpolation unit configured to interpolate biological information in the sampling interval and acquire the biological information in a case where the sampling interval is deviated from a predetermined value.

What is claimed is:

1. A biological-information acquisition apparatus comprising:
a plurality of flexible attachment devices each provided with an electrode that is attached to a body and that is configured to acquire biological information; and
a connector configured to connect the plurality of attachment devices.

2. The biological-information acquisition apparatus according to claim 1, wherein one of the attachment devices is attached to a chest area and acquires an electrocardiographic chest-lead waveform as the biological information.

3. The biological-information acquisition apparatus according to claim 1, wherein one of the attachment devices is attached to a right arm or a left arm and acquires an electrocardiographic limb-lead waveform as the biological information.

4. The biological-information acquisition apparatus according to claim 1, wherein one of the attachment devices is attached to a hip and acquires an electrocardiographic limb-lead waveform as the biological information.

5. The biological-information acquisition apparatus according to claim 1, further comprising:
a main device configured to acquire the biological information from each of the attachment devices and transmit the biological information to a communication apparatus via intra-body communication.

6. The biological-information acquisition apparatus according to claim 5, wherein the main device is connected to one of the attachment devices via the connector.

7. The biological-information acquisition apparatus according to claim 5, wherein the communication apparatus transmits the biological information to an electronic apparatus configured to determine whether each electrode is in an attached state based on the biological information.

8. The biological-information acquisition apparatus according to claim 7, wherein the electronic apparatus includes a display unit configured to display a guide for attaching the attachment devices to the body.

9. The biological-information acquisition apparatus according to claim 1, wherein each electrode is formed by laminating, an adhesive layer attachable to the body, a first conductive layer, an electrolyte layer, and a second conductive layer in this order, and a predetermined potential difference is applied between the first conductive layer and the second conductive layer when the electrode is to be detached from the body.

10. The biological-information acquisition apparatus according to claim 9, wherein the electrolyte layer and the adhesive layer are each composed of a polyethylene-ethylene-oxide-hexamethylene copolymer or SBR polyethylene-oxide copolymer impregnated with an ionic material.

11. The biological-information acquisition apparatus according to claim 9, wherein the first conductive layer and the second conductive layer are each formed of a carbon fiber layer.

12. The biological-information acquisition apparatus according to claim 9, wherein the first conductive layer has a foamable solid material mixed therein.

13. A communication system comprising:
a biological-information acquisition apparatus including an electrode that is attached to a body and that is configured to acquire biological information, a transmitting unit configured to transmit the biological information acquired by the electrode, and a power receiving unit configured to receive supplied electric power; and
an information processing apparatus including a power supply unit configured to perform power supply to the biological-information acquisition apparatus via intra-body communication, a receiving unit configured to receive the biological information from the transmitting unit via intra-body communication, a sampling-interval determination unit configured to determine a sampling interval extending from when the power supply commences to when the biological information is received, and an interpolation unit configured to interpolate biological information in the sampling interval and acquire the biological information in a case where the sampling interval is deviated from a predetermined value.
